(19) United States
(12) Patent Application Publication
(10) Pub. No.: US 2024/0016649 A1
(43) Pub. Date: Jan. 18, 2024

(54) Tinted Nasal Dilator

(71) Applicant: ASO LLC, Sarasota, FL (US)

(72) Inventors: Brooke A. Hagerty, Bradenton, FL (US); Edmund A. Sinda, Bradenton, FL (US)

(21) Appl. No.: 18/221,920

(22) Filed: Jul. 14, 2023

Related U.S. Application Data
(60) Provisional application No. 63/389,880, filed on Jul. 16, 2022.

(51) Int. Cl. A61F 5/08

(52) U.S. Cl. CPC A61F 5/08

(57) ABSTRACT

An external nasal dilator can be adhered across a bridge of a human nose and apply a recoil force to outer walls of nasal passages. The external nasal dilator has a transparent tinted portion that is tinted a first color. Packaging contacting the external nasal dilator is non-transparent and has a second color that contrasts with the first color. The first color is visually perceptible through the transparent tinted portion when the packaging is contacting the external nasal dilator and is visually imperceptible when the external nasal dilator is adhered to the nose.

### CROSS-REFERENCE TO RELATED APPLICATION

This claims the benefit of priority from Application No. 63/389,880, filed Jul. 16, 2022, which is incorporated by reference in its entirety.

### FIELD

This relates to the field of nasal dilators and, more particularly, to external nasal dilators.

### BACKGROUND

External nasal dilators help with breathing through the nose. They gently expand the nasal passages by applying an outward force that prevents the nostrils from collapsing inwardly when the wearer inhales. Such dilators are composed of a resilient band of material attached to an adhesive substrate. When the dilator is adhered to the skin of the nose the resilient band acts as a spring and applies an outward recoil force to hold the left and right nostrils open while breathing.

### BRIEF SUMMARY

Although external nasal dilators are very popular and effective, there is a need to improve them by providing additional functionality beyond merely applying mechanical forces to the nostrils. Nasal dilators having various decorative colored components are already known. The problem with these nasal dilators is that the colored components are specifically designed to be visible to others when someone is wearing the nasal dilator. As discussed herein, color may also be used to help users select from different types of nasal dilators, but many nasal dilator users would prefer the nasal dilator to be more discrete when worn. The nasal dilators described here advantageously have a color-tinted portion with a color that is visible when the dilator is on its packaging, but becomes visually imperceptible to the average human when someone is wearing the nasal dilator.

An example of such a nasal dilator is configured to adhere across a bridge of a human nose and apply a recoil force to outer walls of nasal passages of the nose. The nasal dilator has a transparent tinted portion that is tinted a first color, and it is in contact with packaging that is non-transparent and has a second color that contrasts with the first color. The first color is visually perceptible through the transparent tinted portion when the packaging is contacting the nasal dilator, but it is visually imperceptible when the dilator is adhered across the bridge of the nose.

The nasal dilator may also include one or more of the following features.

The transparent tinted portion may have an opacity of between 0.1% and 20%.

The transparent tinted portion may have an opacity of between 0.5% and 10%.

The nasal dilator may include a plurality of components laminated together. The plurality of components may include a cover layer, base layer, and a resilient layer between the cover layer and base layer.

The second color may be a shade of white.

The transparent tinted portion may have an opacity such that the first color is visually imperceptible on Fitzpatrick scale Type II skin.

The packaging may include a wrapper, a release liner, or both.

An example of a method includes forming an external nasal dilator configured to adhere across a bridge of a human nose and apply a recoil force to outer walls of nasal passages of the nose. The nasal dilator has a transparent tinted portion that is tinted a first color. The opacity of the transparent tinted portion is selected such that the first color is visually imperceptible through the transparent tinted portion when the nasal dilator is on non-transparent packaging but is visually imperceptible when the dilator is removed from the packaging and adhered across the bridge of the nose.

The method may also include one or more of the following features.

The transparent tinted portion may have an opacity of between 0.1% and 20%.

The transparent tinted portion may have an opacity of between 0.5% and 10%.

The dilator may include a plurality of components laminated together. The plurality of components may include a cover layer, base layer, and a resilient layer between the cover layer and base layer.

The packaging may be a shade of white.

The opacity may be further selected such that the first color is visually imperceptible on Fitzpatrick scale Type II skin.

The packaging may include a wrapper, a release liner, or both.

An example of a device includes a nasal dilator with a base layer with an adhesive for adhering the dilator to a human nose. The dilator includes a resilient layer that applies a recoil force to the nose when it is adhered to the nose. The dilator includes a cover layer over the resilient layer. The base layer, resilient layer, and cover layer are laminated together. The dilator includes a transparent tinted portion that is tinted a first color and has an opacity of between 0.1% and 20%. The device also includes packaging contacting the dilator, which is non-transparent and a shade of white. The first color is visually perceptible through the transparent tinted portion when the packaging is contacting the dilator but is visually imperceptible when the dilator is adhered to the nose.

The device may also include one or more of the following features.

The transparent tinted portion may have an opacity of between 0.5% and 10%.

The opacity may be such that the first color is visually imperceptible on Fitzpatrick scale Type II skin.

The packaging may include a wrapper, a release liner, or both.

The base layer, cover layer, and resilient layer may be transparent.

In certain examples, the base layer, cover layer, and resilient layer are transparent and the transparent tinted portion forms part of only one of the base layer, cover layer, or resilient layer.

### BRIEF DESCRIPTION OF THE DRAWINGS

FIG. 1 is a diagram of an example of a tinted external nasal dilator being worn on a wearer's nose.

FIG. 2 is front view of the nasal dilator of FIG. 1 without a release liner.

FIG. 3 is a back view of the nasal dilator of FIG. 1 without a release liner.

FIG. 4 is front view of the nasal dilator of FIG. 1 with a release liner.

FIG. 5 is a back view of the nasal dilator of FIG. 1 with a release liner.

FIG. 6 is an exploded side view of an example of the nasal dilator.

FIG. 7 is an exploded side view of an example of the nasal dilator.

FIG. 8 is an exploded side view of an example of the nasal dilator.

FIG. 9 is a front view of the nasal dilator of FIG. 1 on its packaging.

FIG. 10 is a front view of another example of the nasal dilator.

FIG. 11 is a front view of another example of the nasal dilator.

FIG. 12 is a front view of another example of the nasal dilator.

FIG. 13 is a front view of another example of the nasal dilator.

### DETAILED DESCRIPTION

This disclosure describes certain examples, but not all possible examples of the nasal dilator and related methods. Where a particular feature is disclosed in the context of a particular example, that feature can also be used, to the extent possible, in combination with and/or in the context of other examples. The nasal dilator and related methods may be embodied in many different forms and should not be construed as limited to only the features and examples described here.

The nasal dilator described here improves upon existing nasal dilator designs because it includes a tinted portion that provides a color contrast with the packaging material on which the dilator is packaged. This tinted portion is visible when the dilator is placed against the contrasting packaging, but is substantially visually imperceptible when worn on a human nose. This function assists the wearer with identifying the type of nasal dilator the wearer is selecting when the wearer opens the packaging, but, when worn on the nose, the tinted portion is substantially visually imperceptible to other people, making the nasal dilator more discrete.

External nasal dilators exist in many different shapes and sizes. A relatively simple nasal dilator construction is shown in the drawings for ease of reference. It should be understood, however, that the advantageous tinted feature is not solely useful on nasal dilators constructed as shown here. It may also be applied to other types and designs of conventional nasal dilators.

Throughout the drawings, surface shading is used to represent transparency, opaqueness, color tint, and color contrast. Diagonal surface shading with respect to the shaded surface indicates the surface is transparent. Horizontal surface shading with respect to the surface indicates the surface is non-transparent, which means translucent or opaque. Dotted shading indicates the shaded surface is tinted with color and the tinted color is visually perceptible to a human.

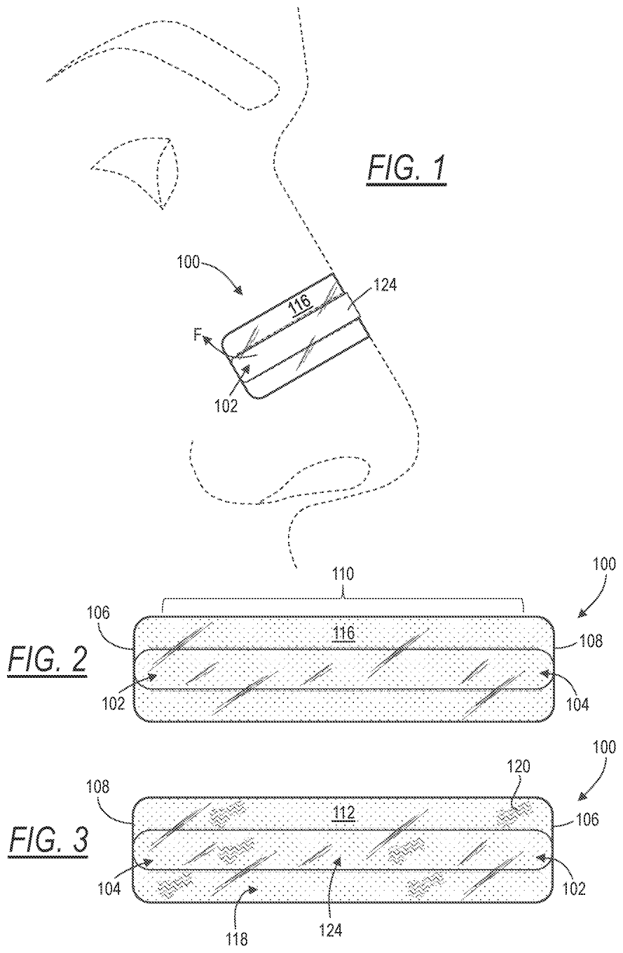

Referring to FIG. 1, an example of the external nasal dilator 100 is shown adhered to the outer wall of a human nose. When adhered to the nose, the nasal dilator 100 applies a recoil force F to the outer wall, which lifts the outer wall tissue of the nostrils and dilates the internal nasal passages. Dilating the internal nasal passages reduces airflow resistance and increases airflow through the nasal passages when breathing. The right side of the nose is a mirror image of FIG. 1.

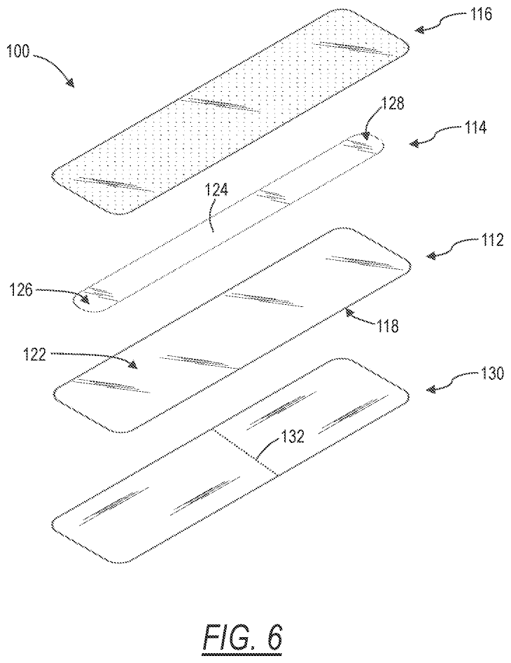

In FIGS. 2-5, the nasal dilator 100 example of FIG. 1 is shown removed from the nose. FIGS. 6-8 show three exploded views of three different examples of the nasal dilator 100 in which a different component is tinted.

Referring to FIGS. 2 and 3, the nasal dilator 100 has an elongated shape and exists in a substantially planar state when not being worn by a wearer. The nasal dilator 100 extends laterally between a first end region 102 and a second end region 104 that, respectively, define a left outer lateral edge 106 and right outer lateral edge 108 of the nasal dilator 100. The first end region 102 and second end region 104 are designed to affix the nasal dilator 100 to opposed sides of the nose over the wearer's nasal passages.

The nasal dilator 100 also includes an intermediate region 110 that extends laterally between the first end region 102 and second end region 104. The intermediate region 110 is designed to traverse the bridge of the wearer's nose between the first end region 102 and second end region 104.

Details of the nasal dilator 100 construction will now be described with reference to FIGS. 2-8. The nasal dilator 100 may be composed of a plurality of laminated layers including a base layer 112, resilient layer 114, and cover layer 116.

The base layer 112 and cover layer 116 may be composed of thin, flexible material that is comfortable. Such a material may include, for example, woven or non-woven fabric, such as polyester, polyethylene, polypropylene, polyurethane, or the like. In the alternative, the material may include a plastic woven or non-woven material, such as polyethylene, polypropylene, polyurethane, or the like. In other examples, the material may be a foam, silicone, or the like.

The base layer 112 includes a nose engagement side 118 having adhesive 120 thereon for adhering the nasal dilator 100 to the nose. In certain examples the adhesive 120 covers substantially the entire nose engagement side 118.

The adhesive 120 may be an adhesive material suitable for skin contact. The adhesive material is selected from skin contact adhesives that can adhere firmly to the skin, but are removable without damaging the skin. Examples of skin contact adhesives include, acrylic adhesives, silicone adhesives, hydrogels, hydrocolloids, silicone or the like. The adhesive material may also include an additive that provides benefits to the skin such as vitamins, vitamin E, and/or zinc oxide, for example.

The adhesive 120 is transparent, which allows the light to pass through it and allows the color of the background on which it is placed to be visible through it.

The base layer 112 includes a resilient layer engagement side 122 adapted to engage with the resilient layer 114 such that the resilient layer 114 cooperatively mates with the base layer 112. The resilient layer engagement side 122 may include adhesive or the like that is capable of attaching the resilient layer 114 to the base layer 112.

In some particular examples, the base layer 112 has a thickness of about 0.5 to about 500 μm. The base layer 112 may be oxygen and/or moisture permeable or impermeable, depending on the materials chosen.

The resilient layer 114 includes at least one resilient member 124 that extends laterally between the first end region 102 and second end region 104. The resilient member 124 may extend completely to the left and right lateral edges 106, 108 or may terminate in the first end region 102 and second end region 104 short of the left and right lateral edges 106, 108.

The resilient layer 114 may include a single resilient member 124, or multiple resilient members 124. In the example shown, a single resilient member 124 extends in the lateral direction across the nasal dilator 100. The resilient member 124 has a first end 126 and a second end 128.

Resilient members 124 may be made from any suitable material having the desired flexibility and resiliency to generate the desired amount of recoil force, such as metal, plastic, or the like. The recoil force is the spring biasing force created by the resiliency of the resilient members. The resilient members are typically flat, semi-rigid, and resilient so as to generate a recoil force when flexed across the nose, which is what lifts the outer wall tissues of the nostrils. This recoil force is typically between about 15 and 35 grams, or about 25 grams. In a particular example, the resilient member 124 is made of biaxially oriented polyester with a thickness of 0.1 mm to 0.3 mm and a width of 0.3 mm to 0.6 mm. The thickness of the resilient member 124 may be selected to obtain a nasal dilator with the desired amount of recoil force. Other examples of materials from which a resilient member 124 can me made include polyethylene, polypropylene, or the like.

The resilient member 124 may be secured to the resilient layer engaging side 122 of the base layer 112 by adhesive material thereon. The adhesive material may cover the resilient layer engaging side 122 or may be substantially the same size and shape as the resilient member 124.

The number and design of the resilient member(s) 124 may depend on the desired amount of force to be applied to the nose, the direction of force to be applied to the nose, and aesthetic considerations.

Resilient members 124 are disposed between the base layer 112 and cover layer 116 and held in place by an adhesive, which also stops the base layer 112 and cover layer 116 from separating when the nasal dilator 100 is being worn or removed.

In certain examples, the resilient member 124 is transparent, which allows light to pass through it and allows the color of the background on which it is placed to be visible.

The cover layer 116 is primarily made of a thin, flexible material. The cover layer 116 may be made of the same material as the base layer 112 or a different material from the base layer 112. The cover layer 116 and base layer 112 may be laminated together using adhesive and/or a thermally fusible film. The cover layer 116 may have a thickness of about 1 to about 500 μm and may be oxygen and moisture permeable or impermeable.

When the nasal dilator is adhered to the wearer's nose, the recoil force from the resilient member 124 draws the outer wall tissue of the left and right nasal passages outward. The flexibility of the base layer 112, resilient layer 114, and cover layer 116 all allow the nasal dilator 100 to substantially conform to the unique curves of the nose of each individual wearer.

In certain examples, the cover layer 116 and base layer 112 are transparent, which allows light to pass through them and allows the color of the background on which they are placed to be visible from the front of the dilator 100.

When the cover layer 116, resilient member 124, base layer 112, and skin contact adhesive 120 are transparent, light can pass through them, allowing the color of the surface on which the dilator 100 is located to be visible from the front of the dilator 100.

In the example of FIGS. 2 and 3, the cover layer 116, resilient member 124, and base layer 112 are transparent. The cover layer 116, base layer 112, and/or resilient member 124 are tinted with a color. FIGS. 2 and 3 do not show a release liner so that the nose engagement side 118 and adhesive on the base layer 112 are visible.

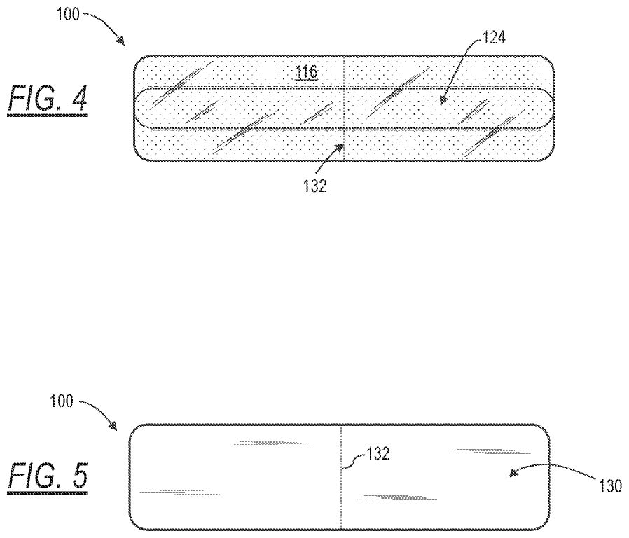

In FIGS. 4-8 the nasal dilator 100 includes a release liner 130 attached to the nose engagement side 118. The release liner 130 forms part of the nasal dilator 100 packaging and is designed to cover the adhesive 120 prior to use. Before the wearer applies the nasal dilator 100 to the nose, the wearer removes the release liner 130 to expose the adhesive 120 and then adheres the dilator 100 onto the nose.

The release liner 130 may have a single piece construction that covers the adhesive 120 or a two-piece construction with a seam 132 proximal to the center of the dilator 100 as shown.

The release liner 130 is a paper or plastic film used to prevent the adhesive 120 from prematurely adhering. The release liner 130 may be composed of a base material that is coated on one or both sides with a release agent that facilitates easy removal from the adhesive 120. The release liner 130 may be made of any conventional nasal dilator release liner material, such as polymer-coated paper, for example.

The release liner 130 may be opaque or translucent, such that the color of the release liner 130 is visible through the transparent layers of the dilator 100 on the release liner 130. In this case, the release liner 130 provides a background contrast to the tinted portion of the nasal dilator 100 so that the color of the tinted portion is visible when the release liner 130 is attached to the dilator 100.

The release liner 130 may alternatively be transparent in certain other examples.

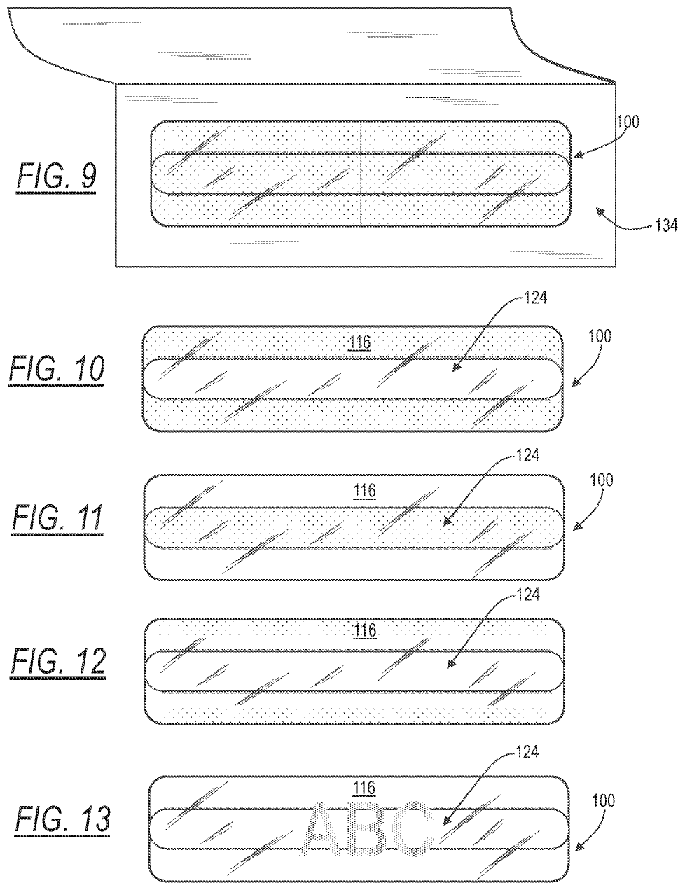

Referring to FIG. 9, the nasal dilator 100 is shown in its wrapper 134, which is used to contain the nasal dilator 100. The wrapper 134 is partially open so the nasal dilator 100 is visible. In certain examples, the wrapper 134 is opaque or translucent, such that the color of the wrapper 134, is visible through the layers of the dilator 100 on top of the wrapper 134 if no release liner 130 is present or if the release liner 130 is transparent. The packaging 134 may provide a background contrast to the tinted portion of the dilator 100 so that the tinted portion is visible when the dilator 100 is in the wrapper 134.

The wrapper 134 may be made of a conventional paper or plastic material in which nasal dilators are packaged.

The tinted portion of the nasal dilator 100 is tinted with a color that contrasts with the color of the release liner 130 and/or wrapper 134.

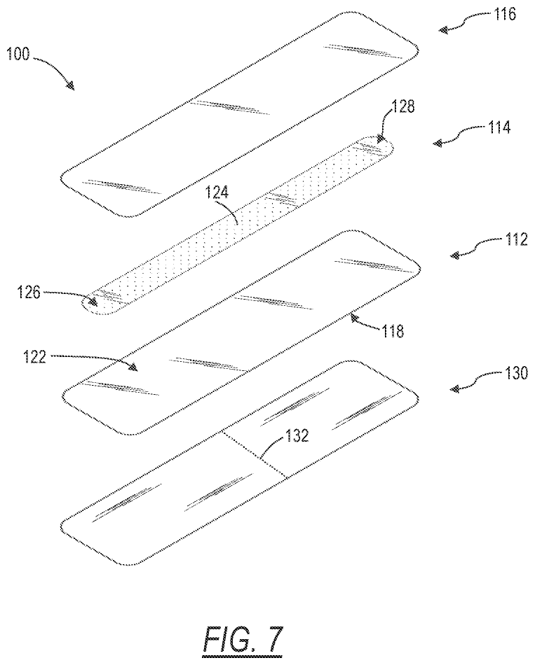

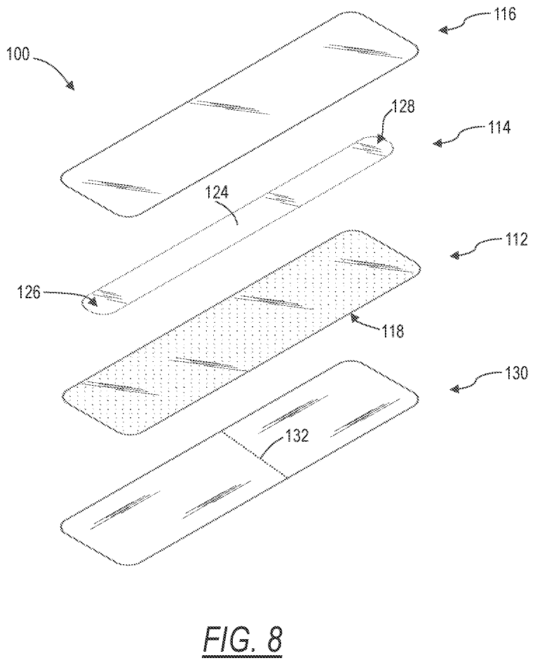

Different components of the nasal dilator 100 may be tinted as shown in the examples of FIGS. 6-8. In FIG. 6, the nasal dilator 100 includes a transparent tinted cover layer 116, a transparent non-tinted resilient layer 114, a transparent non-tinted base layer 112, and an opaque or translucent release liner 130. In FIG. 7, the nasal dilator 100 includes a transparent non-tinted cover layer 116, a transparent tinted resilient member 124, a transparent non-tinted base layer 112, and non-transparent release liner 130. In FIG. 8, the nasal dilator 100 includes a transparent non-tinted cover layer 116, a transparent non-tinted resilient layer 114, a transparent tinted base layer 112, and a non-transparent release liner 130.

The tinted portion of the nasal dilator 100 may otherwise be the skin contact adhesive 120, or the adhesive laminating the resilient member 124 to the cover layer 116 and base layer 112.

The unique tinted aspect of the nasal dilator 100 is apparent when comparing FIG. 1 with FIGS. 4 and 9. In FIG. 1, the nasal dilator 100 is on the nose and the tint of the tinted portion is not visually perceptible, which is why the nasal dilator is not shaded with dots. The skin color of the nose is visible through the nasal dilator 100 because the nasal dilator 100 is transparent. In FIGS. 4 and 9, the tinted portion contrasts with the background color of the release liner 130 and wrapper 134, respectively, and is visible. Although not required, the release liner 130 and/or packaging on which the dilator 100 is located will typically be a shade of white.

The are many possible ways to provide a tint color to the tinted component. The tint color may be applied during manufacturing of the tinted component such that the color is within the tinted component; a colored film may be applied to the tinted component; a color may be painted, printed, or otherwise applied to a surface of the tinted component; or the like.

For the tinted component to retain its transparency and become visually imperceptible when the dilator 100 is being worn, the tinted portion of the nasal dilator 100 is only slightly tinted. If the degree of tint is too high, the tinted portion will be visually perceptible when on the nose. For this reason, the degree of tint may be such that it is as low as possible yet still be visually perceptible on the opaque or translucent background surface.

The degree of tint may be expressed in terms of opacity, which is a measure of the penetrability of visible light through a substrate. A substrate with 0% opacity is completely transparent whereas a substrate with 100% opacity is completely opaque. Opacity may be determined by measuring the transmittance of visible light through the substrate with the opacity being 100%-Transmittance.

Examples of the nasal dilator 100 may have an opacity when measured through the tinted portion of 0.1% to 20%, 0.1% to 15%, 0.1% to 10%, 0.1% to 5%, 0.5% to 20%, 0.5% to 15%, 0.5% to 10%, 0.5% to 5%, 1% to 20%, 1% to 15%, 1% to 10%, or 1% to 5%.

The opacity of a nasal dilator 100 is selected to be visually perceptible on the background surface of the wrapper 134 and or release liner 130, which will typically be a shade of white, while also being visually imperceptible when worn on a wearer's nose against the wearer's skin. Because there are many different skin colors, the opacity may be selected for different skin colors. The opacity may also be selected based on an average skin color of a particular geographic region or group of prospective users of the nasal dilator 100.

Skin colors have been arranged on a scale called the Fitzpatrick scale, which scientifically classifies skin colors from Type I to Type VI. Fitzpatrick, T. B, Arch Dermatol, Vol. 124, 869-871(1988).
Type I—always burns, never tans (palest)
Type II usually burns, tans minimally (light colored but darker than Type I)
Type III—sometimes mildly burns, tans uniformly (golden honey or olive)
Type IV—burns minimally, tans well (moderate brown)
Type V—very rarely burns, tans very easily (dark brown)
Type VI never burns (deeply pigmented dark brown to darkest brown)

In certain examples, the opacity of the nasal dilator 100 is selected so that the tint of the tinted portion is visually imperceptible on Fitzpatrick scale Type II and/or Type I skin colors. If the tint of the tinted portion is visually imperceptible on Fitzpatrick scale Type II and/or Type I skin colors, it will also be visually imperceptible on Type IV-VI skin colors.

The nasal dilator 100 may be designed so that the tinted portion is placed on different locations of the nasal dilator 100. FIGS. 10-13 show four additional examples.

In the nasal dilator 100 example of FIG. 10, the tinted portion is omitted from the surface overlapping the resilient member 124.

In the nasal dilator 100 example of FIG. 11, the tinted portion is on the resilient member 124.

In the dilator 100 example of FIG. 12, the tinted portion is visible along only the outer perimeter of the dilator 100.

In the dilator 100 example of FIG. 13, the tinted portion is a visual indicators such as symbols, designs, letters, or the like.

Every component of the nasal dilator 100 does not need to be transparent. Some portions of the nasal dilator 100 may be translucent or opaque while the tinted portion is located on a transparent portion of the nasal dilator 100.

The nasal dilators 100 described here are different than conventional nasal dilators that have colored components because the colored components of the conventional dilators are designed to be visually perceptible when worn on the nose.

The nasal dilators 100 may be useful to help people identify different types or brands of nasal dilators from among an assortment of nasal dilators. Different types or brands of nasal dilators may be tinted different colors or have different tinting patterns on them for ease of identification. But many people do not want to wear a nasal dilator with colorful features that are noticeable by others. Using a tinted nasal dilator 100 described here will permit people to see the tinted color in the packaging, but the tinted color will be visually imperceptible when worn.

As used herein, visual perceptibility refers to the ability of an average adult human to perceive the tinted color of the tinted portion.

When a feature of the nasal dilator 100 is referred to herein as being “non-tinted,” this means the tinted color of the tinted portion of the nasal dilator 100 is not present in the non-tinted portion. In certain examples of the nasal dilator 100, the non-tinted portions may be substantially colorless and transparent.

A method of making an external nasal dilator includes forming an external nasal dilator configured to adhere across a bridge of a human nose and apply a recoil force to outer walls of nasal passages of the nose. The external nasal dilator has a transparent tinted portion that is tinted a first color. The above-described external nasal dilators are possible examples of the external nasal dilator in this method. Examples of how such external nasal dilators may be formed are described above.

The opacity of the transparent tinted portion is selected such that the first color is visually perceptible through the transparent tinted portion when the external nasal dilator is on non-transparent packaging of the external nasal dilator and is visually imperceptible when the external nasal dilator is removed from the packaging and adhered across the bridge of the human nose.

The desired opacity may be determined by preparing a plurality of tinted portions or completed nasal dilators, then placing each of them over the background color of the packaging to make sure the first color is visible on the packaging. Next, the tinted portions or completed nasal dilators are placed over a human nose or over a background simulating the color of the human nose to make sure the first color is visually imperceptible by the average human against the skin color of the human nose. The tinted portions or nasal dilators that meet both of these criteria may be selected for use in this method.

The nasal dilator and related methods are not limited to the details and features described in connection with the example embodiments. There are numerous variations and modifications of the dilator that may be made without departing from the scope of what is claimed.

That which is claimed is:

1. A device comprising:
an external nasal dilator configured to adhere across a bridge of a human nose and apply a recoil force to outer walls of nasal passages of the human nose, the external nasal dilator having a transparent tinted portion that is tinted a first color; and
packaging contacting the external nasal dilator, the packaging being non-transparent and having a second color that contrasts with the first color;
wherein the first color is visually perceptible through the transparent tinted portion when the packaging is contacting the external nasal dilator and is visually imperceptible when the external nasal dilator is adhered across the bridge of the human nose.

2. The device of claim 1, wherein the transparent tinted portion has an opacity of 0.1%-20%.

3. The device of claim 1, wherein the transparent tinted portion has an opacity of 0.5%-10%.

4. The device of claim 1, wherein the external nasal dilator includes a plurality of components laminated together, the plurality of components including a cover layer, base layer, and a resilient layer between the cover layer and base layer.

5. The device of claim 1, wherein the second color is a shade of white.

6. The device of claim 1, wherein the transparent tinted portion has an opacity and the opacity is such that the first color is visually imperceptible on Fitzpatrick scale Type II skin.

7. The device of claim 1, wherein the packaging includes a wrapper and/or a release liner.

8. A method comprising:
forming an external nasal dilator configured to adhere across a bridge of a human nose and apply a recoil force to outer walls of nasal passages of the nose, the external nasal dilator having a transparent tinted portion that is tinted a first color; and
an opacity of the transparent tinted portion being selected such that the first color is visually perceptible through the transparent tinted portion when the external nasal dilator is on non-transparent packaging of the external nasal dilator and is visually imperceptible when the external nasal dilator is removed from the packaging and adhered across the bridge of the human nose.

9. The method of claim 8, wherein the transparent tinted portion has an opacity of 0.1%-20%.

10. The method of claim 8, wherein the transparent tinted portion has an opacity of 0.5%-10%.

11. The method of claim 8, wherein the external nasal dilator includes a plurality of components laminated together, the plurality of components including a cover layer, base layer, and a resilient layer between the cover layer and base layer.

12. The method of claim 8, wherein the packaging is a shade of white.

13. The method of claim 8, wherein the opacity is further selected such that the first color is visually imperceptible on Fitzpatrick scale Type II skin.

14. The method of claim 8, wherein the packaging includes a wrapper and/or a release liner.

15. A device comprising:
an external nasal dilator having (a) a base layer with an adhesive for adhering the external nasal dilator to a human nose; (b) a resilient layer that applies a recoil force to the human nose when adhered thereto; (c) a cover layer over the resilient layer, the base layer, resilient layer, and cover layer being laminated together; and (d) a transparent tinted portion that is tinted a first color, the transparent tinted portion having an opacity of 0.1%-20%; and
packaging contacting the external nasal dilator, the packaging being non-transparent and a shade of white;
wherein the first color is visually perceptible through the transparent tinted portion when the packaging is contacting the external nasal dilator and is visually imperceptible when the external nasal dilator is adhered to the human nose.

16. The device of claim 15, wherein the transparent tinted portion has an opacity of 0.5%-10%.

17. The device of claim 15, wherein the opacity is such that the first color is visually imperceptible on Fitzpatrick scale Type II skin.

18. The device of claim 15, wherein the packaging includes a wrapper and/or a release liner.

19. The device of claim 15, wherein the base layer, cover layer, and resilient layer are transparent.

20. The device of claim 15, wherein the base layer, cover layer, and resilient layer are transparent and the transparent tinted portion forms part of only one of the base layer, cover layer, or resilient layer.
